(19) United States
(12) Patent Application Publication
(10) Pub. No.: US 2017/0153431 A1
(43) Pub. Date: Jun. 1, 2017

(54) HIGH-DEFINITION PARTICLE DETECTION DURING CENTRIFUGATION

(71) Applicant: ZebraSci, Inc, Temecula, CA (US)

(72) Inventors: Hoang Thanh Nguyen, Riverside, CA (US); Jaan Noolandi, La Jolla, CA (US); Robert James Schultheis, Temecula, CA (US)

(21) Appl. No.: 15/362,640

(22) Filed: Nov. 28, 2016

Related U.S. Application Data
(60) Provisional application No. 62/261,847, filed on Dec. 1, 2015.

(51) Int. Cl. G02B 21/00; B04B 13/00; G01N 33/15; G02B 21/06; G02B 21/36

(52) U.S. Cl. CPC G02B 21/0016; G02B 21/06; G02B 21/361; G02B 21/362; G02B 21/365; G01N 33/15; B04B 13/00; B04B 2013/006

(57) ABSTRACT

High-definition particle detection during centrifugation of a pharmaceutical liquid is provided. Centrifugation of fluid containers drives particles to the interior surface of the container if the particles are denser than the fluid and to the middle of the container if the particles are less dense than the fluid. The imager can then be focused directly on the particle itself for rapid identification without the need for computing complex particle trajectories. If the centrifugation of the container is carried out at an angle to the axis of symmetry of the container, particles can be driven to a single line on the interior surface of the container by the centrifugal force, making the identification of the particles even more straightforward than in two dimensions. The particle imager can also be attached to the rotating container to prevent blurring of the particle image due to the relative motion of the container and imager.

### CROSS-REFERENCE TO RELATED APPLICATIONS

This application claims priority from U.S. Provisional Patent Application 62/261,847 filed Dec. 1, 2015, which is incorporated herein by reference.

### FIELD OF THE INVENTION

This invention relates to particle detection methods and systems during centrifugation.

### BACKGROUND OF THE INVENTION

It is important to identify and characterize different types of particles, which may be present as impurities in a solution, which contains a drug product. Unfortunately, visual inspection cannot detect particles below a certain size (about 100 microns) and in any case is time consuming when dealing with large numbers (millions) of containers.

Hence automated inspection systems such as the one described in the Amgen patent application (US2014/0177932) have been developed. In the Amgen system, which includes computer tracking software and imaging hardware, once the imager identifies a particle, a complex computer program estimates the particle trajectory from reversed time-series data and then identifies the particle based on its characteristic trajectory. Since several cc's of fluid volume must be scanned to detect and identify particles, significant data compression and processing is required to estimate the particle trajectory which leads to uncertainty in the characterization of the particle trajectory. The present invention addresses these problems and issues.

### SUMMARY OF THE INVENTION

The present invention provides a method and system for high-definition particle detection during centrifugation of a pharmaceutical liquid that overcomes at least some of the problems and issues in the art. High-definition is defined as high magnification with a shallow depth of field. The system involves a container, a light source, a motor, an imaging sensor and an optical device. The container is filled with the pharmaceutical liquid. Examples of containers are a syringe, a vial, a cartridge or an ampoule. The light source illuminates the pharmaceutical liquid in the container. Various light patterns can be applied such as, but not limited to, a low-angle dark field, a collimated dark field, a diffused dark field, a collimated bright field, or a diffused bright field.

The imaging sensor is situated capable of imaging the reflected light that reflects off the illuminated pharmaceutical liquid. The optical device is optically aligned with the imaging sensor to focus and magnify the reflected light reflected off the pharmaceutical liquid onto the imaging sensor. The optical device magnifies the particles in the pharmaceutical liquid 2 to 20 times. The motor is spins the pharmaceutical liquid in the container and applies a centrifugal force at a certain rpm (ranging from 1000 to 2000 rpm) onto the particles in the pharmaceutical liquid. The G-force is equal to 1.12×R×(RPM/1000)², where R is the radius of rotation in mm, which might be helpful to determine the required duration for particles of varying sedimentation coefficients.

The optical device and imaging sensor are connected to the motor so that when the motor spins both the optical device and imaging sensor spin at the same rpm around the container. The imaging sensor images the static and/or dynamic behavior of the particles in the pharmaceutical liquid within the container during the application of the centrifugal force. In one variation of the system and method, a mechanism can be added for changing the angle of the container with respect to the motor during centrifugation (i.e. tilting the container during rotation/spinning). This allows for control of the orientation of the inner-wall in relation to the axis of rotation. One could make the particles travel up or down the container by tilting the container outward or inward, respectively. This mechanism is useful because while one may be able to view the particles in high definition, it may be difficult to differentiate the particles from surface defects on the container. Manipulating the position of the particles by tilting would be undeniable proof one is observing free-floating particles.

In the present invention it is shown that moderate centrifugation of fluid containers (up to 2000 RPM) drives particles to the interior surface of the container if the particles are denser than the encompassing fluid (usually an aqueous solution) and to the middle of the container if the particles are less dense than the encompassing fluid. The imager can then be focused directly on the particle itself for rapid identification without the need for computing complex particle trajectories. Furthermore if the centrifugation of the container is carried out at an angle to the axis of symmetry of the container, particles can be driven to a single line on the interior surface of the container by the centrifugal force, making the identification of the particles even more straightforward than in two dimensions. The particle imager can also be attached to the rotating container to prevent blurring of the particle image due to the relative motion of the container and imager.

Advantages of embodiments of the invention are for example rapid identification of particles on the inside wall of the (centrifuged) container by direct imaging in the case of particles more dense than the solution and by direct imaging in the middle of the (centrifuged) container in the case of particles less dense than the solution. This allows for the use of high magnification and a shallow field of focus to identify the nature and origin of the particles (glass flakes from a delaminating container, pieces of dust and dirt from the container filling process, or aggregates of drug molecules from the formulation process). In addition, the use of an imager rotating with the container allows for clear pictures which help to identify the particles.

### BRIEF DESCRIPTION OF THE DRAWINGS

FIG. 1A shows a schematic of the system setup according to an exemplary embodiment of the invention for on-axis rotation of the specimen. Element 1 is a container with a pharmaceutical fluid. Element 2 is a light source. Element 3 is a motor for spinning. Element 4 is an optical element for focusing and magnifying the reflected light on imaging sensor 5. Element 6 is a connection between the motor 3 and imaging sensor 5 (including optical device 4) to ensure spinning at the same speed.

FIG. 1B shows a schematic of the system according to an exemplary embodiment of the invention for off-axis rotation of the specimen. Element 1 is a container with a pharmaceutical fluid. Element 2 is a light source. Element 3 is a motor for spinning. Element 4 is an optical element for focusing and magnifying the reflected light on imaging sensor 5. Element 6 is a connection between the motor 3 and imaging sensor 5 (including optical device 4) to ensure spinning at the same speed. Element 7 is a joint which controls the angle of the group of Elements 1, 2, 4, and 5 with respect to the axis of Element 3. Element 8 is a counterweight to increase stability of the system.

FIG. 1C shows the schematic of the system setup according to an exemplary embodiment of the invention shown in FIG. 1A with the addition of Element 9, which is a mechanism for changing the angle of the container with respect to the motor during centrifugation.

FIGS. 2A-B show a top-down schematic of the system according to an exemplary embodiment of the invention configured with collimated (FIG. 2A) and diffused (FIG. 2B) dark field lighting. Element 10 is a collimated light source and Element 11 is a diffused light source.

FIGS. 3A-B show a top-down schematic of the system according to an exemplary embodiment of the invention configured with collimated (FIG. 3A) and diffused (FIG. 3B) bright field lighting.

FIG. 4 shows according to an exemplary embodiment of the invention an image of particulates in a glass vial containing aqueous solution, laid on its side for an hour and imaged from the bottom. FIG. 4 shows particles, which have undergone sedimentation. FIG. 4 shows an example of how a high-magnification (e.g., 4×) shallow depth of field lens can resolve these particles that have sedimented to the bottom of the container (note that the container is resting horizontally on its side in this figure with the lens pointed up at the container. This is relevant to the centrifugation use case because the particles will exhibit similar sedimentation behavior, but accelerated (FIG. 4 involved a sedimentation for hours, whereas one could achieve a similar effect via centrifugation in only a few seconds as per the objectives of the invention).

FIG. 5 shows according to an exemplary embodiment of the invention an image of vial containing aqueous solution (control).

FIG. 6 shows according to an exemplary embodiment of the invention an image of fresh micelle solution immediately after loading vial and spinning at 1600 RPM for 3 seconds.

FIG. 7 shows according to an exemplary embodiment of the invention an image of aged micelle solution after 4 days, spun at 1600 RPM for 3 seconds.

FIG. 8 shows according to an exemplary embodiment of the invention the presence of lysozyme protein crystals in the vial 4 days after initial mixing (without spinning).

### DETAILED DESCRIPTION

### Variable-Angle Centrifuge Microscope Images Using Particle Tracking

In this invention, we use a microscope for analyzing free-floating particulate matter in primary containers during active centrifugation. Unlike traditional particle detection systems, which perform inspection after agitating a container, the system described herein performs inspection during centrifugation. This applies a centrifugal force to the container, which pushes free-floating particulate matter to the outer wall of the container. Image sequences are then captured at timed intervals to inspect free-floating particles rendered stationary against the container inner wall due to the centrifugal force.

Unlike US2014/0177932, in the present invention image capture and analysis is performed during centrifugation, rather than after. This applies a completely different dynamic to free-floating particulate matter:
The magnification of optics used in spin and brake inspection systems are often limited by the amount of depth of field required. Since a larger depth of field is required (to visualize particles at any depth in the container), lower magnification optics must be used (e.g., 0.114× to 1.0×). Since particulate matter is forced to the outer edges of the container, this permits a very shallow depth of field required to visualize particulate matter.
Particulate matter becomes stationary once sedimentation has stabilized. For the duration of centrifugation, large particulate matter remains stationary along the inner wall of the container. This permits high-magnification analysis of said particles.

Rotating the camera with the container minimizes motion blur. Due to the high surface speed of the container, pixel blur will be present in any image captures, e.g., via photo multiplier tubes or lower exposure times. Unlike a stationary ocular detector, motion blur caused by centrifugation will not be present.

The method described herein significantly reduces the required depth of field of a particle detection system. This permits the usage of optics with magnification on par with flow microscopy systems (e.g., 2× to 20×). Unlike flow microscopy systems (which require a primary container specimen be emptied and deposited through a flow cell) the system described herein is non-destructive to a primary container specimen.

Dark field illumination reduces the impact of variable fill levels in primary container specimens. If containers have different fill levels and one uses bright field illumination, the resulting images may vary dramatically from one another because the size of an air gap can affect the results. In addition, the light undergoes additional distortion when passing through the meniscus. In a dark field setup, one can selectively observe just the reflected light on the inner wall, for instance, without worrying about the size of any air gap.

The movement of sediment particles on the outer wall of the container can be manipulated to move along the wall by actively varying the angle of the container during centrifugation. The movement of particles can be observed with a camera whose focus adjusts relative to this angle.

### Apparatus Set-Up (Centrifugation and Lighting)

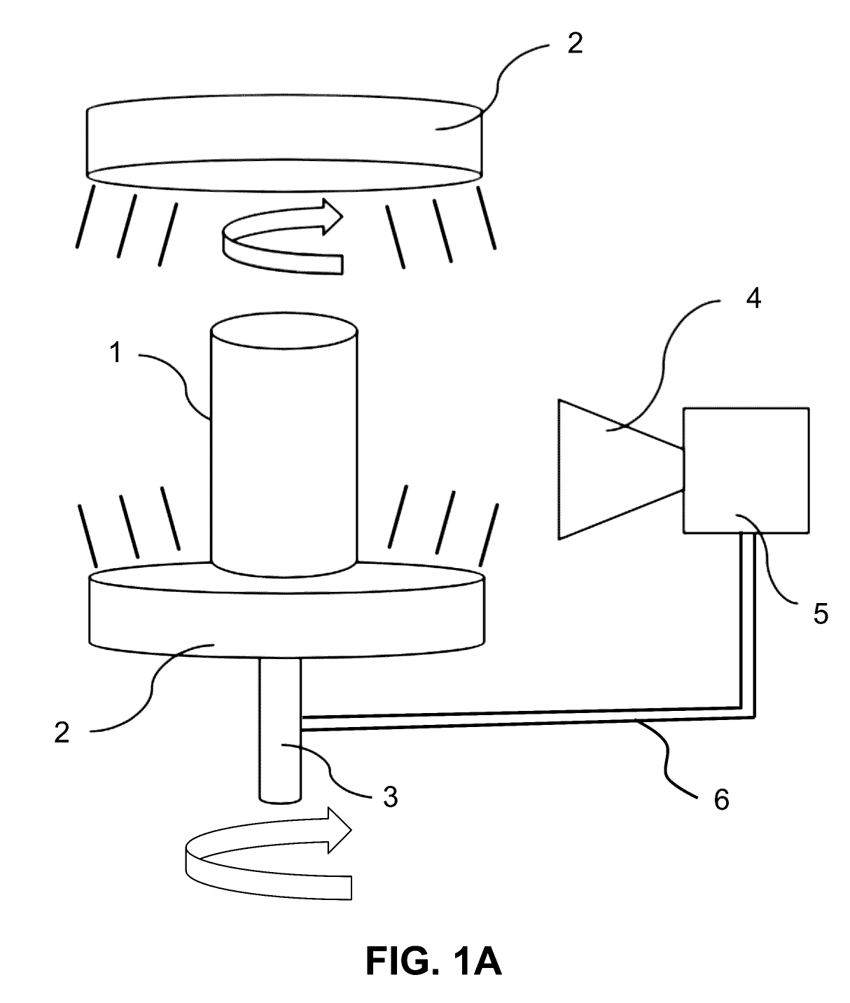

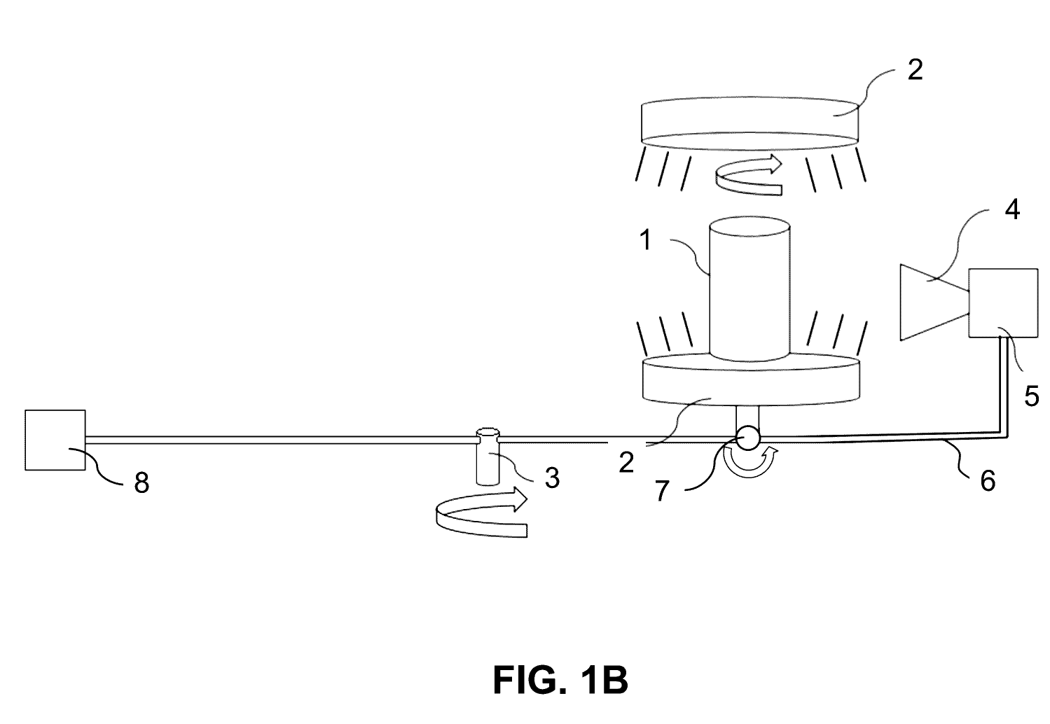

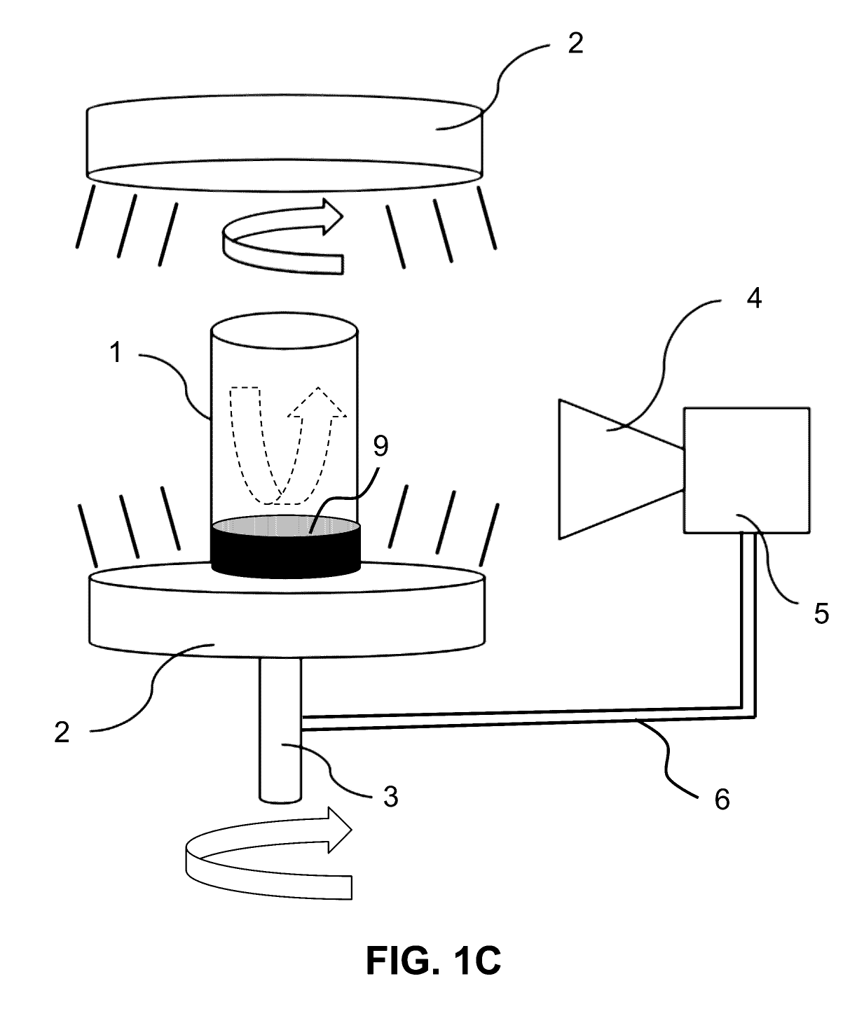

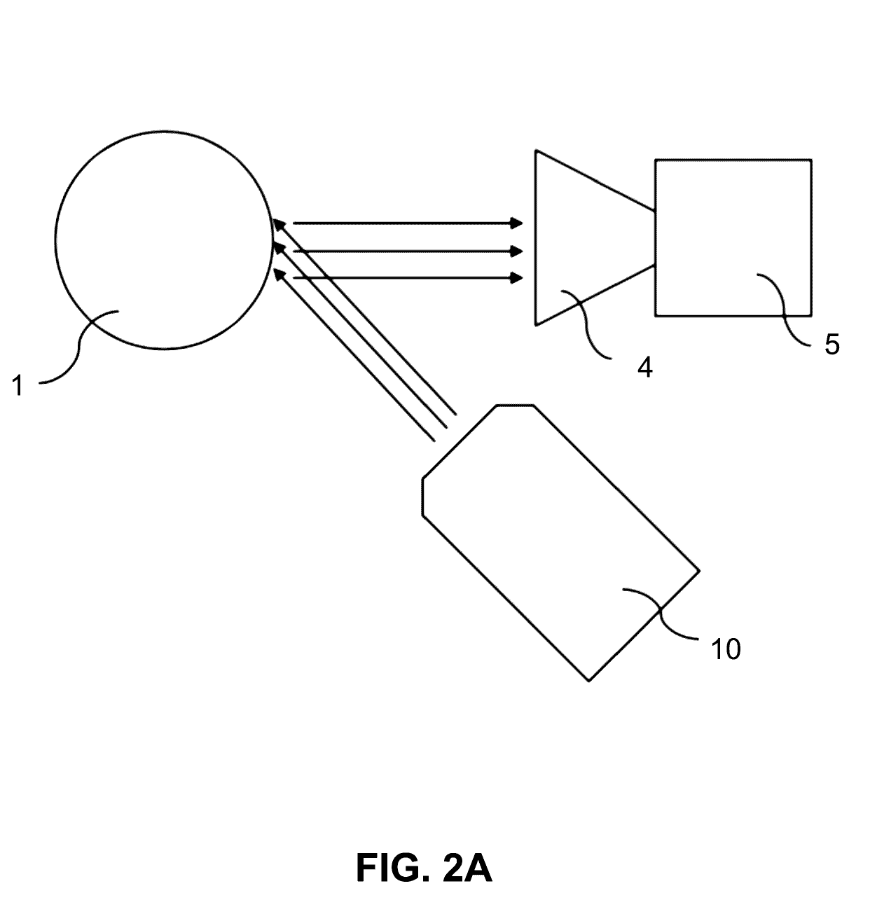

On-axis centrifugation This configuration rotates the part on-axis. That is, free-floating particulates are forced to distribute across the entire inner container wall (FIG. 1A). A variable-angle mechanism can also be added (FIG. 1C) which varies the angle of the container and camera with respect to the axis of rotation.
Off-axis centrifugation This configuration rotates the part off-axis. That is, free-floating particulates are forced to a single side of the inner container wall (FIG. 1B). A variable-angle mechanism (e.g., mechanical joint) varies the angle of the container and camera setup with respect to the axis of rotation. This allows further manipulation of free-floating particulates by forcing them to either the top or bottom of the container, thereby making them easier to distinguish from container surface defects (i.e., the particles can be slowly manipulated up and down the container wall, making them more easily distinguishable from the container wall).

### Lighting

Low-Angle Dark Field This configuration describes a lighting setup where low-angle light is used to illuminate the specimen such that 0th order light rays do not reach the imaging sensor (FIG. 1A) (0th order light rays are not diffracted by the specimen and contribute to background noise).

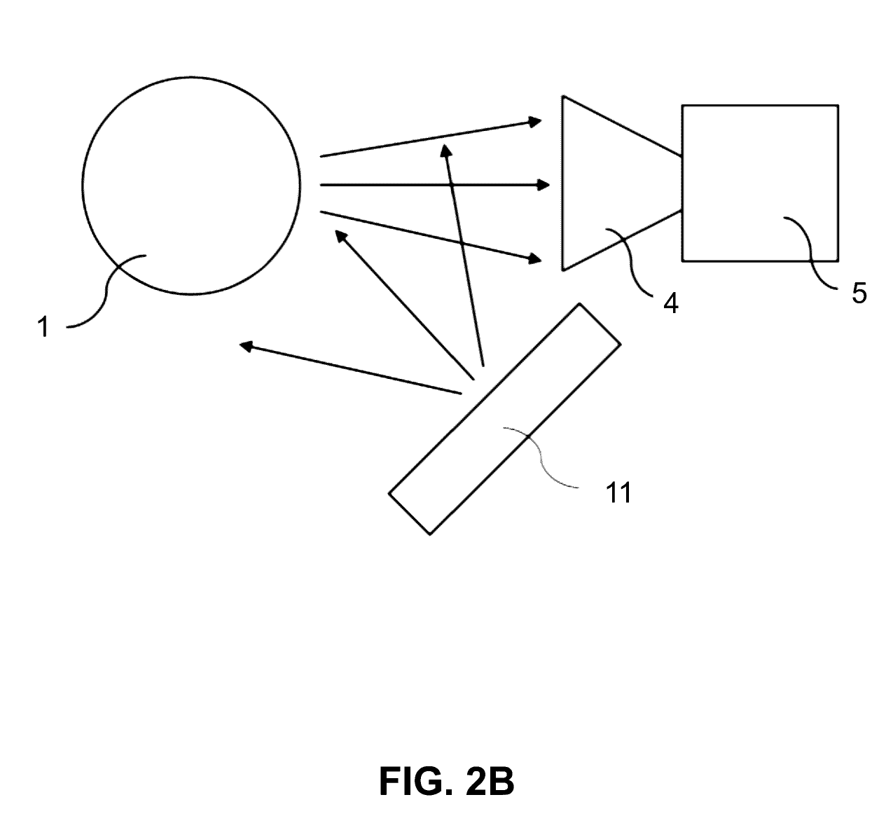

Collimated Dark Field This configuration describes a lighting setup where collimated light is used at an angle such that 0th order light rays do not reach the photo sensor (FIG. 2A).

Diffused Dark Field This configuration describes a lighting setup where diffused light is used at an angle such that 0th order light rays do not reach the photo sensor (FIG. 2B).

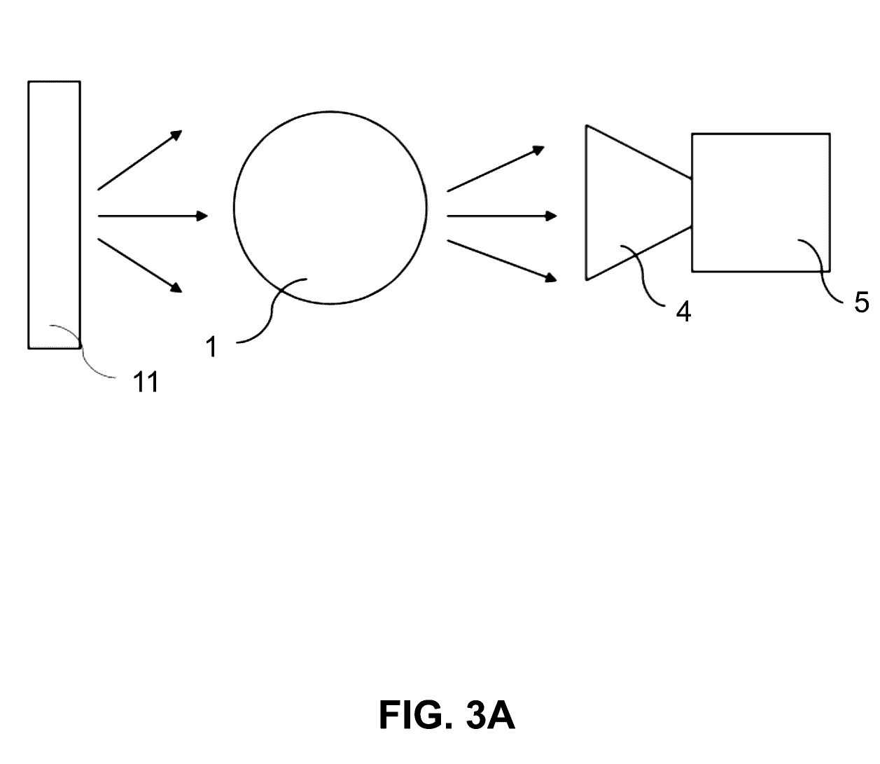

Collimated Bright Field This configuration describes a lighting setup where collimated light is used as a backlight such that all diffracted orders of light rays reach the photo sensor (FIG. 3A).

Diffused Bright Field This configuration describes a lighting setup where diffused light is used as a backlight such that all diffracted orders of light rays reach the photo sensor (FIG. 3B).

### Container

The container used for all experiments was the BD Hypak™ Glass Prefillable Syringe with Fixed Needle (1 ml container). Becton, Dickinson and Company, 1 Becton Drive Franklin Lakes, N.J. 07417-1880.

Value of Detecting Aggregation and/or Crystallization of Therapeutic Products

Evaluation of therapeutic protein products in the in vivo milieu in which they function (e.g., in inflammatory environments or at physiologic pH) may reveal susceptibilities to modifications (e.g., aggregation and deamidation) that result in loss of efficacy or induction of immune responses. Such information may facilitate product engineering to withstand undesirable effects. Sponsors should consider this information in early product design and in development of improved products. Methods that individually or in combination enhance detection of protein aggregates should be employed to characterize these distinct species of aggregates in a product. One or more such assays should be validated for use in routine lot release, and several of them should be employed for comparability assessments. Methods include, but are not limited to the following: size exclusion chromatography (Wang, et al. 2010), analytical ultracentrifugation (Berkowitz 2006), light scattering techniques (Some 2013), Fourier transformed infrared spectroscopy (Gross and Zeppezauer 2010), and field-flow fractionation (Roda, et al. 2009).

### Experimental Protocols

Lysozyme Solution Protocol—

Lysozymes, also known as muramidase or N-acetylmuramide glycanhydrolase, are glycoside hydrolases. These are enzymes that damage bacterial cell walls and are abundant in a number of animal secretions, such as tears, saliva, as well as human milk, and mucus. They form crystals in buffered aqueous solution as described below: Lysozyme crystals were grown in an aqueous buffered solution of sodium acetate and water. 5 mL of the buffered solution was prepared by mixing 5 mL of distilled water with 0.068 g of sodium acetate (anhydrous form, from Sigma-Aldrich). The buffered solution was mixed with 125 mg Lysozyme (Lysozyme from chicken egg white, Sigma-Aldrich). 5 mL of the resulting solution was measured out and had 0.375 g (7.5% wt) of sodium chloride (NaCl, table salt, distributed by Safeway) to facilitate precipitation and crystallization. The final solution was mixed using a magnetic stirrer for 5 minutes.

Micelle Solution Protocol—

Pluronic F127 or Poly(ethylene oxidel)-poly(propylene oxidel)-poly(ethylene oxide) is a triblock copolymer which is currently used in pharmaceutical companies. It readily forms micelles in aqueous solution. Its chemical formula is 250 gm Pluronic F-127 was obtained from Sigma-Aldrich and mixed with distilled water utilizing the protocol listed below: 1. 0.5 gram of Pluronic F-127 was mixed into 20 mL of distilled water. This was mixed continuously for about 1 hour until all of the Pluronic F-127 had dissolved visually. 2. Pluronic F-127 was then added and allowed to sit/mixed over time until the no more would dissolve into solution (approximately 1 gram). 3. Approximately 5 mL was added to the solution and mixed and then allowed to sit over-night. 4. Upon visual inspection all of the Pluronic F-127 had dissolved, and the solution was separated into small vials for further testing.

### Size of Pluronic F127 Micelles

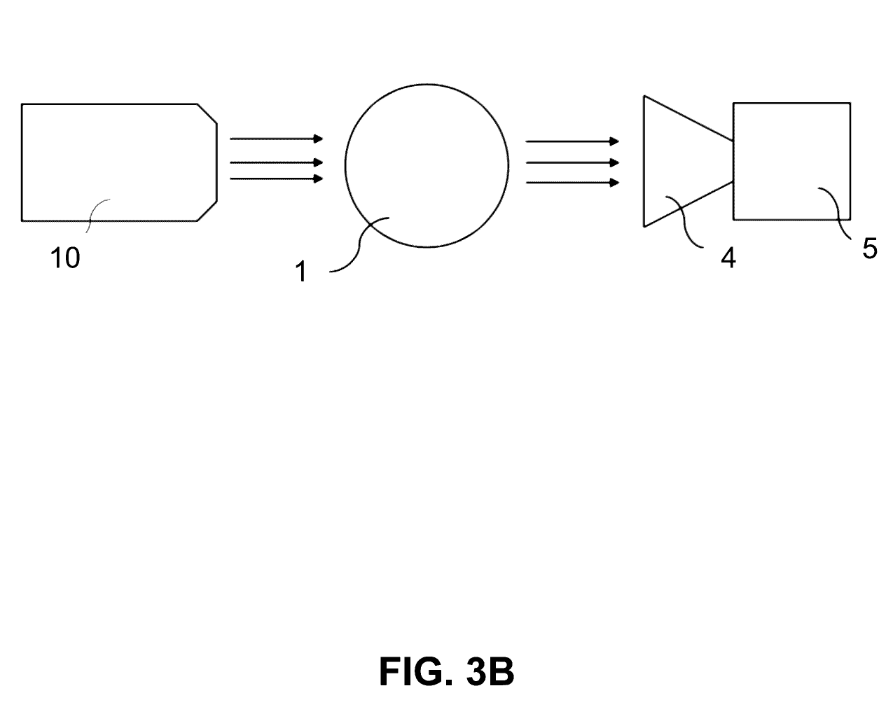

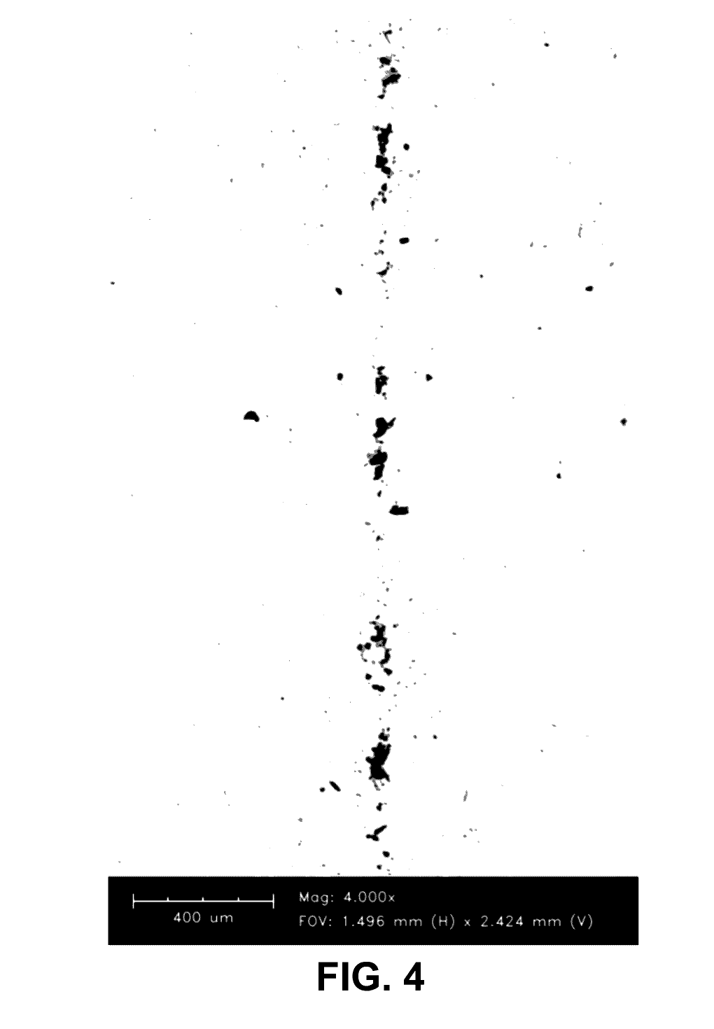

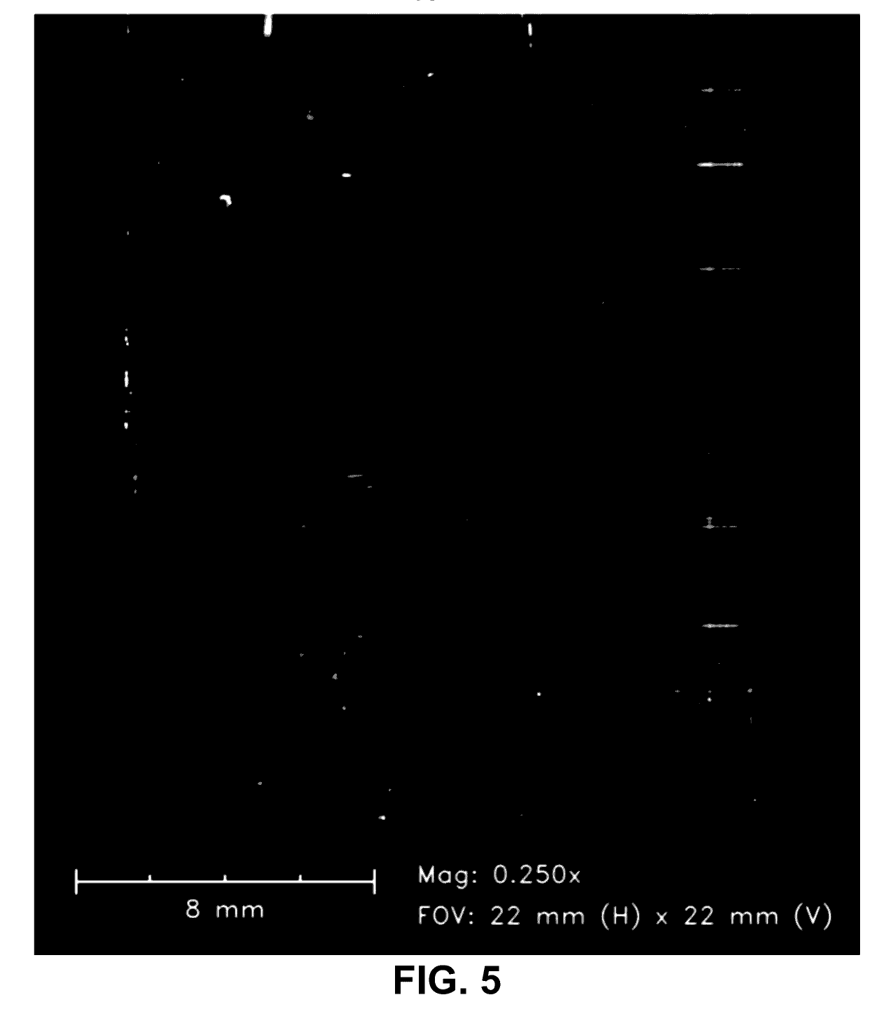

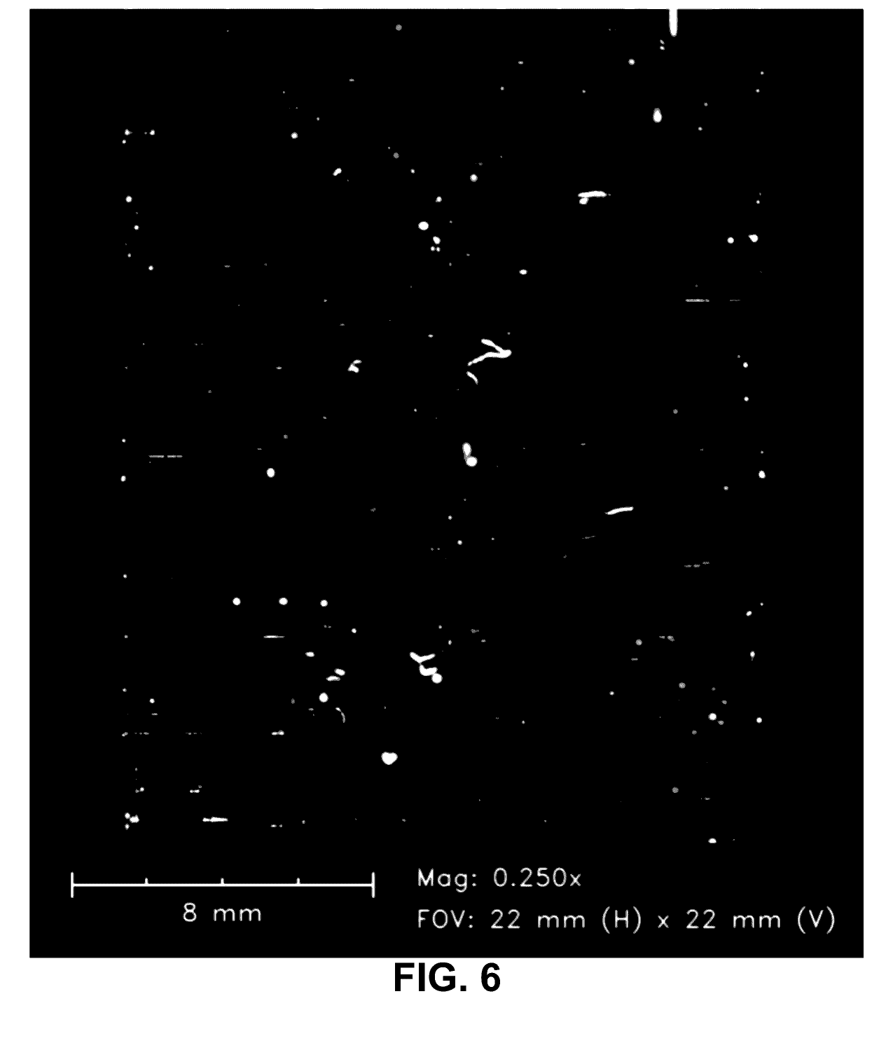

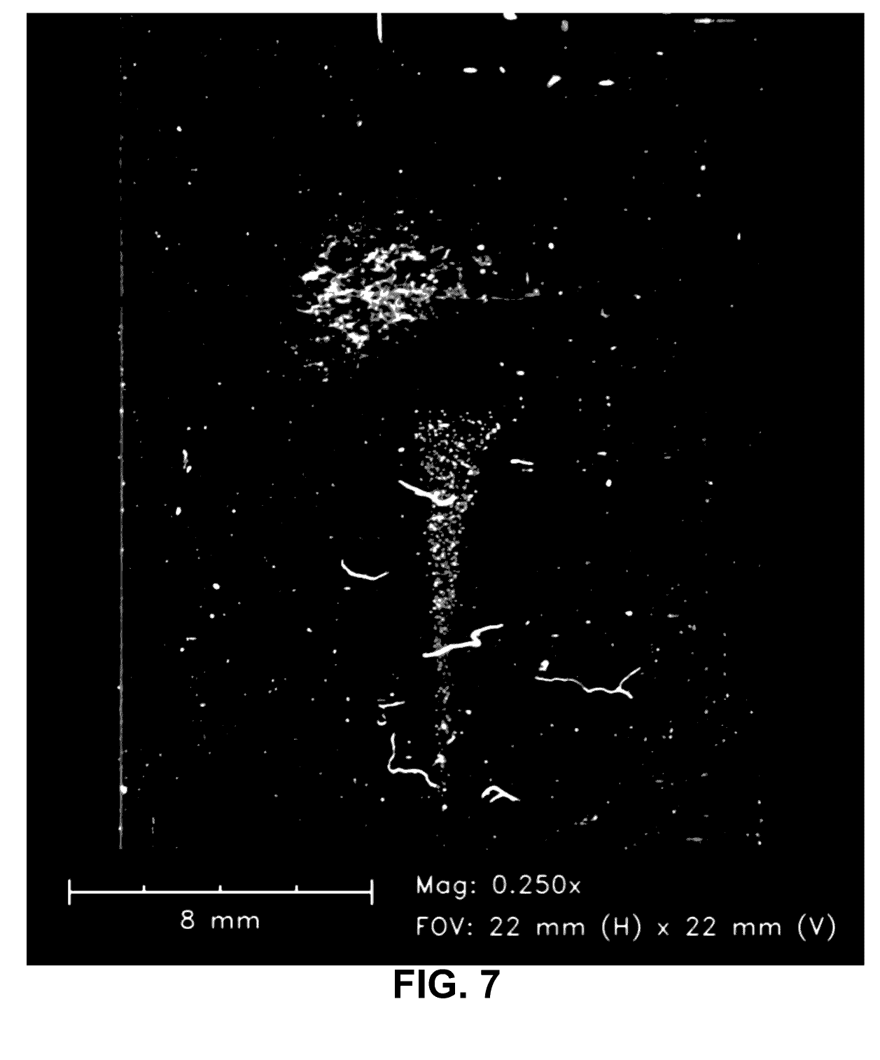

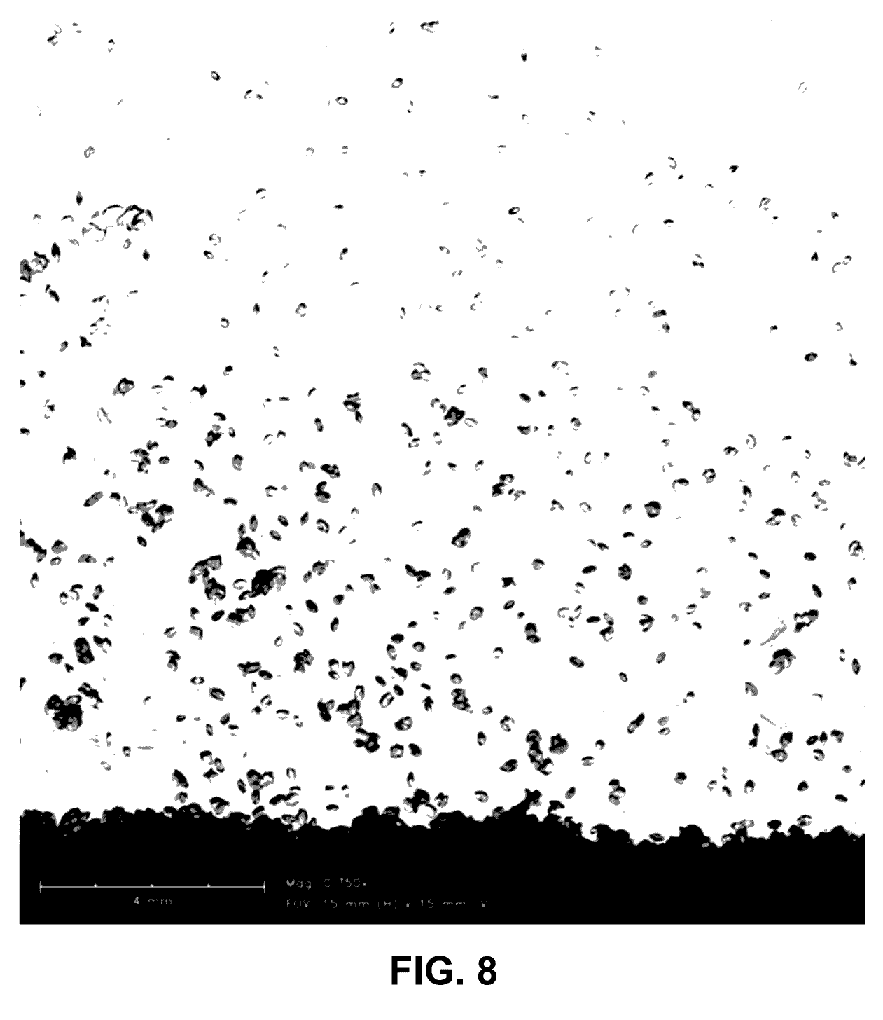

The size of an individual Pluronic F127 micelle is about 10 nanometers (Attwood 1985), which is too small to be detected by normal light scattering techniques (FIG. 4). However the method described in the present invention enables the visualization of aggregates of individual micelles (FIG. 5) after a period of time (in this case 4 days), which proves that a) micelles are present and b) the micelles have aggregated into large clumps which are visible.

### REFERENCES

[Wang, et al. 2010] Wang, Yanwei; Teraoka, Iwao; Hansen, Flemming Y.; Peters, Gunther H., Hassager, Ole. “A Theoretical Study of the Separation Principle in Size Exclusion Chromatography.” Macromolecules, vol. 43, issue 3 (2010): 1651-1659.
[Berkowitz 2006] Berkowitz, Steven A. “Role of Analytical Ultracentrifugation in Assessing the Aggregation of Protein Biopharmaceuticals.” The AAPS Journal 8.3 (2006): E590-E605.
[Some 2013] Some, Daniel. “Light-scattering-based Analysis of Biomolecular Interactions.” Biophysical Reviews, vol. 5, issue 2 (2013): 147-158.
[Gross et al. 2010] Gross, Peter C.; Zeppezauer, Michael. “Infrared Spectroscopy for Biopharmaceutical Protein Analysis.” Journal of Pharmaceutical and Biomedical Analysis, vol. 53, issue 1 (2010): 29-36.
[Roda, et al. 2009] Roda, Barbara; Zattoni, Andrea; Reschiglian, Pierluigi; Moon, Myeong Hee; Mirasoli, Mara; Michelini, Elisa; Roda, Aldo. “Field-flow Fractionation in Bioanalysis: A Review of Recent Trends.” Analytica Chimica Acta, vol. 635, issue 2 (2009): 132-143.
[Attwood et al. 1985] The micellar properties of the ABA poly(oxyethylene)-poly(oxypropylene) block copolymer Pluronic F127 in water and electrolyte solution”. Int. J. Pharmaceutics 26, Issues 1-2, September 1985, Pgs. 25-33.

What is claimed is:

1. A system for high-definition particle detection during centrifugation of a pharmaceutical liquid, comprising:
(a) a container filled with the pharmaceutical liquid;
(b) a light source for illuminating the pharmaceutical liquid in the container;
(c) an imaging sensor for imaging the reflected light off the pharmaceutical liquid;
(d) an optical device optically aligned with the imaging sensor to focus and magnify the reflected light off the pharmaceutical liquid onto the imaging sensor, wherein the optical device magnifies particles in the pharmaceutical liquid 2 to 20 times; and
(e) a motor, connected to the optical device, the imaging sensor and the optical device, for spinning the pharmaceutical liquid in the container and applying a centrifugal force at a certain rpm onto the particles in the pharmaceutical liquid and through the connection simultaneously spinning the optical device and the imaging sensor at the same rpm, wherein the motor spins between a 1000 to 2000 rpm, and wherein the imaging sensor images the static or dynamic behavior of the particles in the pharmaceutical liquid within the container during the application of the centrifugal force.

2. The system as set forth in claim 1, further comprising a mechanism for changing the angle of the container with respect to the motor during centrifugation.

3. The system as set forth in claim 1, wherein the container is a syringe, a vial, a cartridge or an ampoule.
